(19) United States
(12) Patent Application Publication
(10) Pub. No.: US 2013/0046135 A1
(43) Pub. Date: Feb. 21, 2013

(54) DEVICE FOR TREATING OBESITY

(71) Applicant: Paul Super, Solihull West Midlands (GB)

(72) Inventor: Paul Super, Solihull West Midlands (GB)

(73) Assignee: A.M.I. AGENCY FOR MEDICAL INNOVATIONS GMBH, Feldkirch (AT)

(21) Appl. No.: 13/587,238

(22) Filed: Aug. 16, 2012

(30) Foreign Application Priority Data
Aug. 18, 2011 (EP) 11006745.1

(51) Int. Cl. A61F 2/00

(52) U.S. Cl. 600/37

(57) ABSTRACT

A device for treating obesity including a band part (1), which can be applied in a ring shape around the stomach, the band part (1) having a closure mechanism (2, 3) with which the band part (1) can be closed to form a circumferentially closed ring having an opening (4) with a central axis (5), and a support part (17), which is connected to the band part (1) and which protrudes from the latter on one side and provides support for a stomach area lying proximally with respect to the applied band part (1). The support part (17) extends by less than 270° about the central axis (5) in the sectional plane of each cross section that passes through the support part (17) and lies parallel to a mid-plane (6) in which the band part (1) closed to form a ring lies and which extends centrally through the band part.

### CROSS-REFERENCE TO RELATED APPLICATIONS

This application claims the benefit of European Patent Application No. 11 006 745.1, filed Aug. 18, 2011, which is incorporated herein by reference as if fully set forth.

### BACKGROUND

The invention relates to a device for treating obesity, comprising a band part, which can be applied in a ring shape around the stomach, said band part having a closure mechanism with which the band part can be closed to form a circumferentially closed ring having an opening with a central axis, and a support part, which is connected to the band part and which protrudes from the latter on one side and provides support for a stomach area lying proximally with respect to the applied band part.

Gastric bands for treating obesity are known in which a band part is applied in a ring shape around the stomach in order to constrict the stomach and thereby create a narrowed passage for food to pass through the stomach. These gastric bands mostly have a circumferential hollow space that can be filled with a filling medium, such that the size of the opening of the gastric band can be varied by introduction of the filling medium, in order to adjust the constriction of the stomach.

The ingested food builds up in front of this surgically created constriction, and the pressure in the congested portion of the stomach increases, as a result of which the anterior wall of the stomach bulges forwards into the abdominal cavity. In the long term, this excessive forward bulging of the anterior wall of the stomach leads to an undesired widening of the constricted stomach area, which compromises the success of the surgical treatment of obesity.

In order to overcome this disadvantage, EP 1669045 A1 discloses a device of the type mentioned at the outset. The support part mounted on the band part, and protruding from the latter on one side, is applied together with the band part around the stomach and, after the band part has been closed to form a ring, the circumferential ends of the support part now lying adjacent to each other are connected to each other, as a result of which a closed sleeve is formed that circumferentially surrounds an area of the stomach lying proximally from the band part.

Moreover, AT 504158 B1 discloses a device with two or more band parts for constricting the stomach in several stages. Sleeve parts attached to the mutually facing side edges of the band parts extend between the band parts, are applied with the band parts around the stomach and, when the device is in the applied state, envelop the stomach.

US 2005/0119674 A1 and WO 02/053040 A1 disclose further sleeve devices in the form of bands, nets and the like, which are applied circumferentially around the stomach.

Moreover, U.S. Pat. No. 6,165,122 A discloses a perforated elastic sleeve that is placed circumferentially around the heart muscle in order to prevent undesired expansion thereof.

To apply the previously known devices enveloping the stomach, extensive surgical separation of the posterior wall of the stomach from the posterior wall of the abdominal cavity is required. This greatly increases the risk of the operation to the patient. Extensive haemorrhaging can be caused by separation of the tissue layers, a longer operating time is required, and the risk of anaesthesia in this particularly critical group of obese patients is heightened. Furthermore, a considerably larger wound surface remains in the abdominal cavity, associated with increased discharge of wound secretions, which in turn necessitates the use of drainage channels. These medical grounds explain why the known technical solutions, using circumferential and elastic protective devices against undesired enlargement of the surgically constricted stomach areas, have not become widely accepted in surgical practice.

### SUMMARY

The object of the invention is to make available a device of the type mentioned at the outset which counteracts expansion of the stomach in the congested area, wherein the surgical risk during application is reduced. this is achieved by a device with the features of the invention.

The device according to the invention is characterized in that, in the sectional plane of each cross section that passes through the support part and lies parallel to the mid-plane in which the band part closed to form a ring lies and which extends centrally through the band part, the support part extends by less than 270° about the central axis of the opening of the band part. Therefore, in relation to a polar coordinates system lying in the plane of the cross section through the support part and having the central axis as origin, all points of the support part lie in an angle range of less than 270°. Thus, the support part does not extend over the remaining angle range of more than 90°, i.e. no points of the support part lie in this angle range. The support part is therefore circumferentially open, with a circumferential extent of less than 270° along its entire course in the direction of the central axis.

Through this design, surgical separation of the posterior wall of the stomach from the posterior wall of the abdominal cavity is required only to the extent that a passage (channel) is needed for the engagement of the gastric band itself. The support part, which in the applied state covers that part of the stomach lying proximally from the band part in the area of the anterior wall of the stomach, does not have to be guided around the posterior wall of the stomach. However, in order to achieve a stable support of the congested stomach area, the support part can advantageously be connected, in particular by sewing, to the posterior wall of the abdominal cavity at points away from the band part and on both sides (=laterally) of the supported stomach area. Such connection points to the posterior wall of the abdominal cavity preferably lie in areas where a side edge of the support part, which side edge delimits the extent of the support part in the direction of the central axis at the end of the support part remote from the band part, meets the longitudinal edges of the support part, which delimit the extent of the support part about the central axis. The support part is advantageously strengthened, e.g. by fabric inserts, at the connection points. Suture eyelets are preferably formed on the support part at these connection areas or transition areas between the longitudinal edge and the respective side edge. Additional and preferably strengthened connection points, in particular suture eyelets, to the posterior wall of the abdominal cavity can be provided, particularly in the area of the longitudinal edges of the support part. In addition, connection points can also be provided for connecting the support part to the stomach wall, which connection is once again preferably made by sewing, for example in the area of the side edge of the support part.

By means of the design according to the invention, it is possible to combine the advantages of conventional gastric bands without support parts, which bands can be applied in a minimally invasive intervention, and gastric bands with support parts, which bands counteract an expansion of the congested stomach area.

One advantageous embodiment of the invention is characterized in that, in the area of the support part lying at a distance from the mid-plane that is more than two thirds of the maximum distance of the side edge of the support part from the mid-plane, the greatest extent of the support part about the central axis is smaller than the greatest extent of the support part about the central axis in the area of the support part which lies at a distance from the mid-plane amounting to less than one third of the maximum distance of the side edge of the support part from the mid-plane. The extent of the support part about the central axis, at a defined distance from the mid-plane, indicates the angle range about which the support part extends in a cross section lying at this distance from the mid-plane.

This design provides good support of the congested stomach area, the support part being able to be secured to the posterior wall of the abdominal cavity such that the support part reliably performs its supporting function.

The support part is expediently designed with a multiplicity of through-openings distributed across its expanse, wherein the sum of the surface areas of the through-openings preferably take up a majority of the total surface area encompassed by the support part. The support part particularly preferably has a net-like design.

The closure mechanism for closing the band part, in order to form a circumferentially closed ring, can be designed in a conventional manner. In particular, connectable closure parts arranged in the area of the two band ends can be provided for this purpose. In an advantageous embodiment, a first of the two closure parts has a locking projection in a known manner, and the second of the two closure parts has a through-opening through which the first closure part can be drawn, the locking projection of the first closure part engaging with the second closure part behind the through-opening.

To connect the support part to the band part, the support part is preferably adhesively bonded to the band part, preferably in the area of one of the longitudinal edges of the band part.

For the circumferentially open design of the support part, said support part, in the connection area to the band part, does not extend over the entire length of the band part (i.e. not about the entire circumference of the band part closed to form a ring). Preferably, the band part has a first portion of its longitudinal extent that adjoins a first end of the band part and that has a length amounting to at least one third of the total length of the band part, preferably at least two fifths of the total length of the band part, wherein this first portion of the longitudinal extent of the band part is not provided with any support part protruding from one side of the band part. The support part protrudes laterally from the band part only over a second portion of the longitudinal extent of the band part, wherein the second portion of the longitudinal extent of the band part adjoins the second end of the band part and extends over the longitudinal extent of the band part over which the first portion of the band part does not extend.

Since the first portion of the longitudinal extent of the band part is free from laterally protruding support parts, the first portion of the band part can be drawn through a relatively small channel that has been prepared between the posterior wall of the stomach and the posterior wall of the abdominal cavity, such that this first portion extends around the posterior wall of the stomach. Thereafter, the closure mechanism, which advantageously has closure parts at both ends of the band part, can be closed. The support part thus lies in the area of the anterior wall of the stomach and, as has already been mentioned, can then be secured to the posterior wall of the abdominal cavity in areas lying away from the band part laterally on both sides of the stomach.

### BRIEF DESCRIPTION OF THE DRAWINGS

Further advantages and details of the invention are explained below with reference to the attached drawing, in which:

FIG. 1 shows an perspective view of an illustrative embodiment of a device according to the invention, with the band part in the closed state;

FIG. 2 and FIG. 3 show the device from FIG. 1 in perspective views from different viewing directions;

FIG. 4 shows a side view of the device from FIG. 1;

FIG. 5 shows a side view of FIG. 1 from another viewing direction;

FIG. 6 shows a cross section along the line 6-6 from FIG. 5;

FIG. 7 shows a cross section along the line 7-7 from FIG. 5;

FIG. 8 shows a cross section along the line 8-8 from FIG. 5;

FIG. 9 shows a cross section along the line 9-9 from FIG. 5;

FIG. 10 shows a view of FIG. 5 in viewing direction E;

FIG. 11 and FIG. 12 show perspective views of the device from FIG. 1, from different viewing directions, and with the band part in the opened state; and

FIG. 13 shows a schematic depiction of the implanted device with attached injection port.

### DETAILED DESCRIPTION OF THE PREFERRED EMBODIMENTS

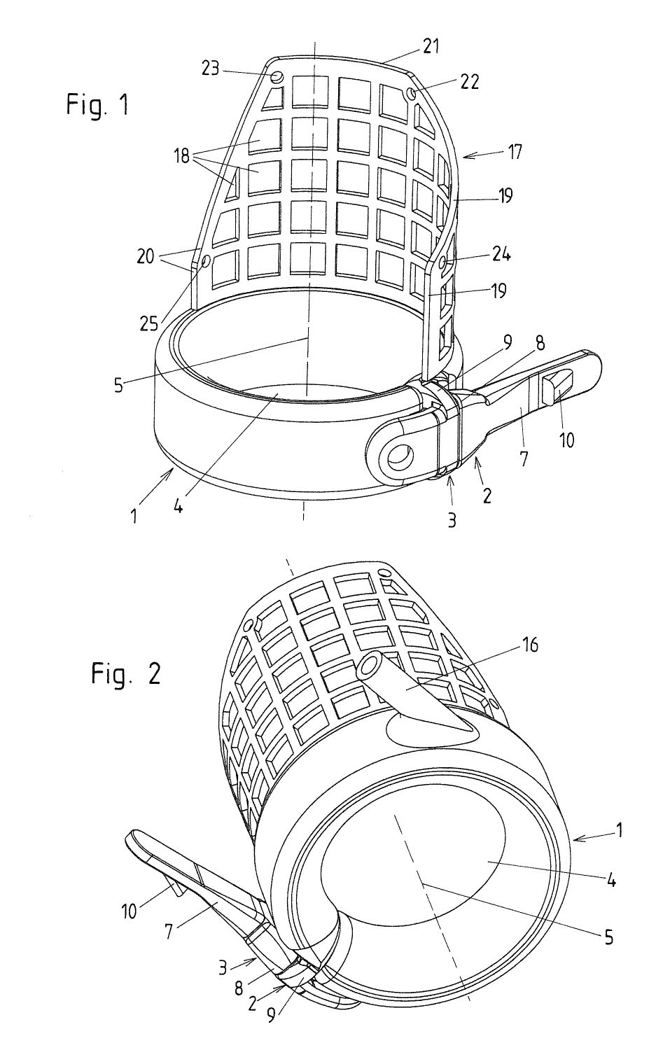

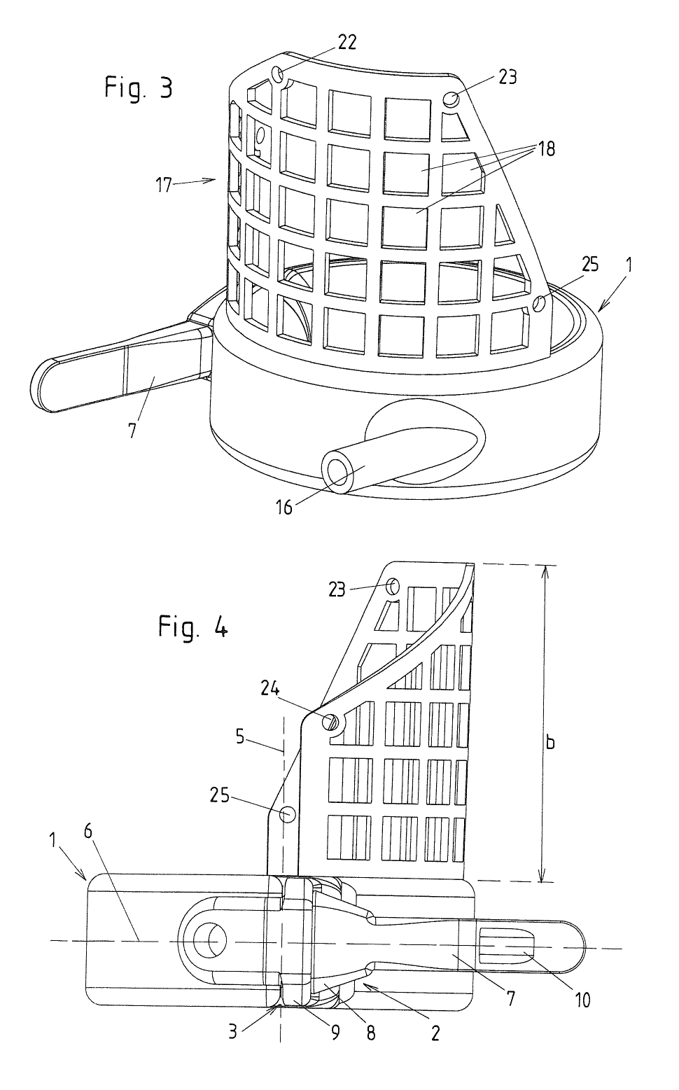

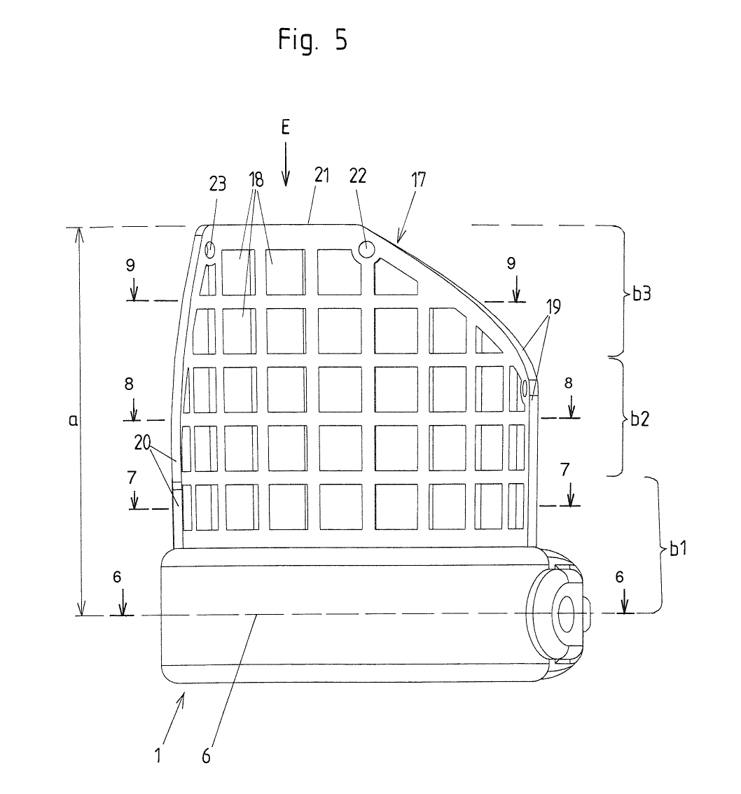

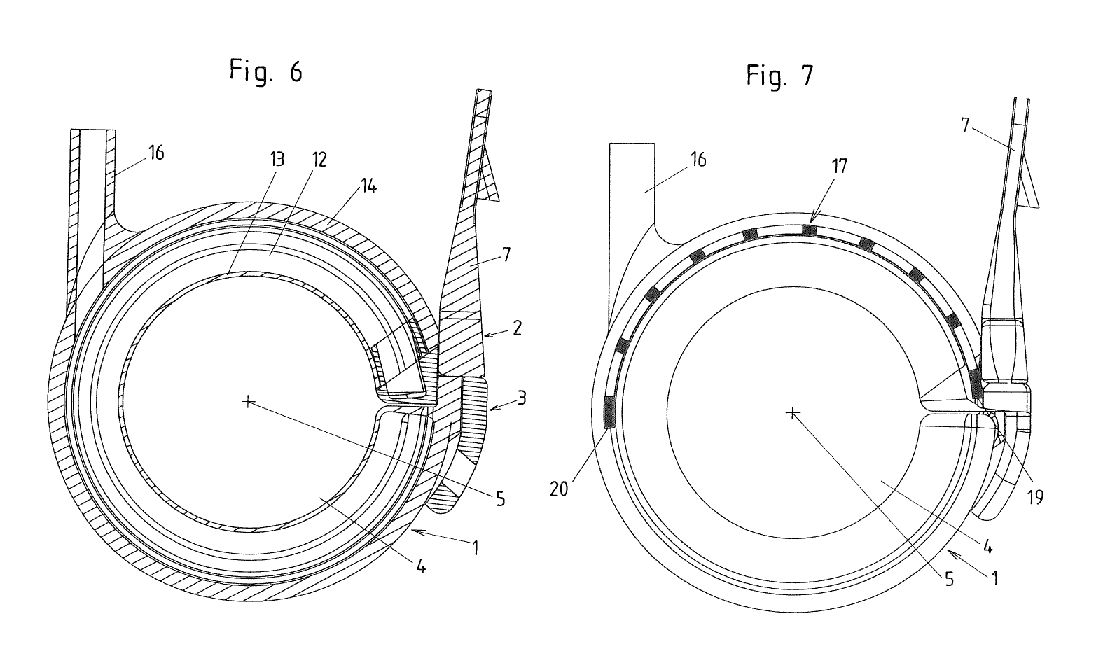

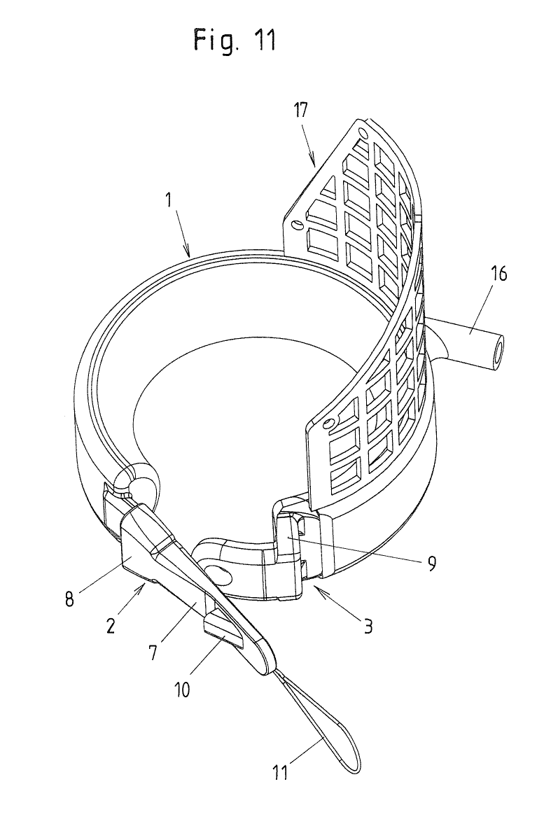

An illustrative embodiment of a device according to the invention is shown in FIGS. 1 to 13. The device comprises a band part 1 having, in the area of its two ends, first and second closure parts 2, 3, which form a closure mechanism for connecting the two ends of the band part 1. In the closed state of the closure mechanism (FIGS. 1 to 10 and 13), a circumferentially closed ring is formed, which surrounds an opening 4 with a central axis 5. The band part 1, closed to form a ring, lies in a mid-plane 6 that passes centrally through the band part 1. In the illustrative embodiment, the band part 1 is mirror-symmetrical with respect to the mid-plane 6. The central axis 5, which passes centrally through the opening 4, is preferably at right angles to the mid-plane 6.

The closed band part 1 forms in particular a circular ring, i.e. extends along a line of a circle.

In the embodiment of the closure mechanism shown, the first closure part 2 has a continuation 7 that protrudes past the front end of the band part 1 and that has a locking projection 8. The second closure part 3 is formed by a web 9, which protrudes outwards near the other front end of the band part 1 and has an insert opening for the continuation 7. The continuation 7, pulled through the insert opening, engages with its locking projection 8 behind the web 9.

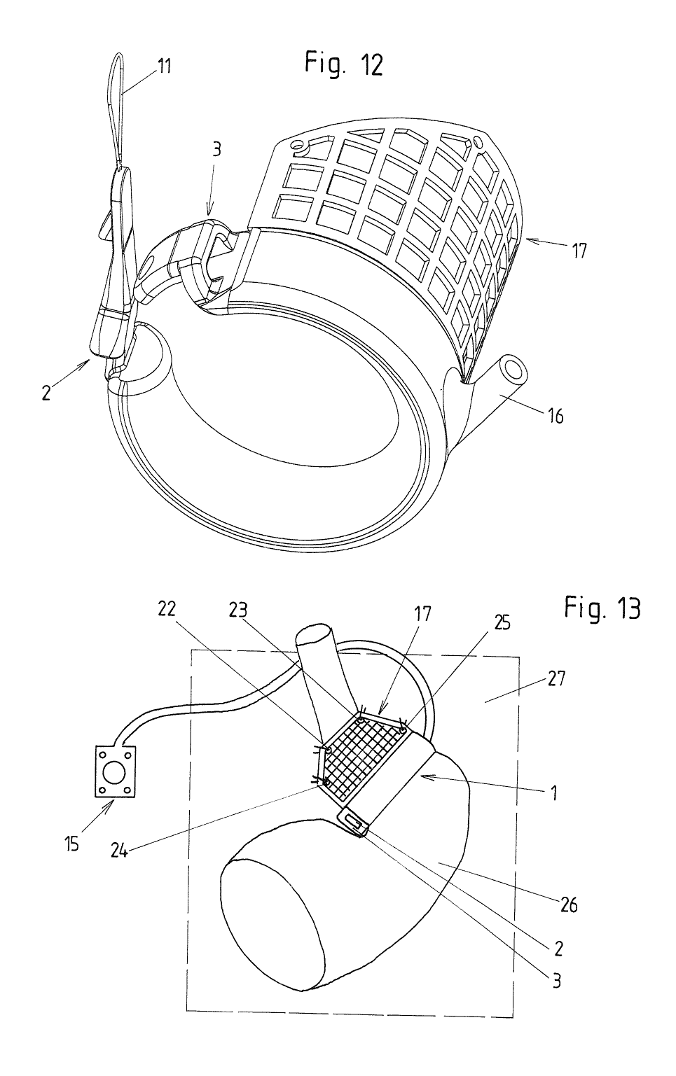

Through the use of a preliminary locking projection 10, a preliminary locking position can be made available when the continuation 7 is partially pulled through. To make it easier for the continuation 7 to be pulled through, it is possible to provide a pulling loop 11, which is shown only in FIGS. 11 and 12.

Closure mechanisms of other designs can also be provided for closing the band part 1.

In its interior, the band part 1 has a hollow chamber 12 which extends in the longitudinal direction thereof, preferably along substantially the entire length thereof, and which can be filled with a filling medium. Upon introduction of a filling medium, an inner area 13 of the band part 1 facing the central axis 5 expands in the direction of the central axis 5, as a result of which the free cross section of the opening 4 can be adjusted. An area 14 of the band part 1 directed away from the central axis 5 can be made less expandable than the inner area 13, for example by means of a fabric insert.

An injection port 15 can be used in a known manner to fill the hollow chamber 12, which injection port 15 is shown schematically in FIG. 13 and is connected by a hose to a connector tube 16 of the band part 1, the inner hollow space of which connector tube 16 opens into the hollow chamber 12.

At least in the area of its outer surface, the band part 1 is made of a biocompatible or biologically inert material, in particular silicone or polyurethane. Preferably, apart from optionally provided reinforcing inserts and the connector tube 16, the band part 1 is composed overall of an elastically extensible material.

A support part 17 is connected to the band part 1, preferably by adhesive bonding, and protrudes laterally from the band part 1, i.e. it extends away from the band part 1 in the direction of the central axis 5. The protruding width b of the support part 17, measured in the direction of the central axis 5, is expediently in the range of 2 cm to 8 cm.

The band part 1 and the support part 17 are designed as a prefabricated unit, i.e. are already connected to each other at the place of manufacture. The support part 17 is preferably adhesively bonded to the band part 1 in the area of one of the longitudinal edges of the latter.

The support part 17 is a planar structure, i.e. its thickness, which is less than 4 mm, preferably less than 2 mm, is substantially smaller than its length and width.

The support part 17 has a multiplicity of through-openings 18 distributed across its expanse. The support part, as shown, preferably has a net-like or grid-like (=net) design. An embodiment as an elastic film with or without punched openings is also conceivable. The preferred size of the through-openings (which can be designated as mesh openings in the design as a net) lies in the range of 1 mm² to 10 mm².

The support part 17 is elastic and is comprised, at least in the area of its outer surface, but preferably throughout, of a biocompatible or biologically inert material, in particular of an elastomer, for example silicone or polyurethane. The hardness of the material of the support part 17 is expediently in the range of 10 to 60 Shore A.

When the band part 1 is in the state in which it is closed to form a ring, the support part 17 extends along only part of the complete circumference about the central axis 5. More precisely, in each cross section through the support part 17 and parallel to the mid-plane 6, the support part 17 extends by less than 270° about the central axis, preferably by less than 225°.

In the illustrative embodiment shown, in the area b3 of the support part 17 in which the distance of the support part 17 from the mid-plane 6 is more than two thirds of the maximum distance a of the support part 17 from the mid-plane 6, the greatest extent of the support part 17 about the central axis 5 is smaller, preferably at least 10% smaller, than the greatest extent of the support part 17 about the central axis 5 in the area b1 of the support part 17, in which the distance of the support part 17 amounts to less than one third of the maximum distance a of the support part 17 from the mid-plane 6.

In the illustrative embodiment shown, in the area b2 of the support part 17 in which the distance of the support part 17 from the mid-plane 6 is more than one third and less than two thirds of the maximum distance a of the support part 17 from the mid-plane 6, the greatest extent of the support part 17 about the central axis 5 is smaller, preferably at least 10% smaller, than the greatest extent of the support part 17 about the central axis 5 in the area b1 of the support part, in which the distance of the support part 17 from the mid-plane 6 amounts to less than one third of the maximum distance a of the support part 17 from the mid-plane 6.

Moreover, in the illustrative embodiment shown, in the area b3 of the support part 17 in which the distance of the support part 17 from the mid-plane 6 is more than two thirds of the maximum distance a, the greatest extent of the support part 17 about the central axis 5 is smaller, preferably at least 10% smaller, than the greatest extent of the support part 17 about the central axis 5 in the area b2 of the support part 17, in which the distance of the support part 17 from the mid-plane 6 amounts to more than one third and less than two thirds of the maximum distance.

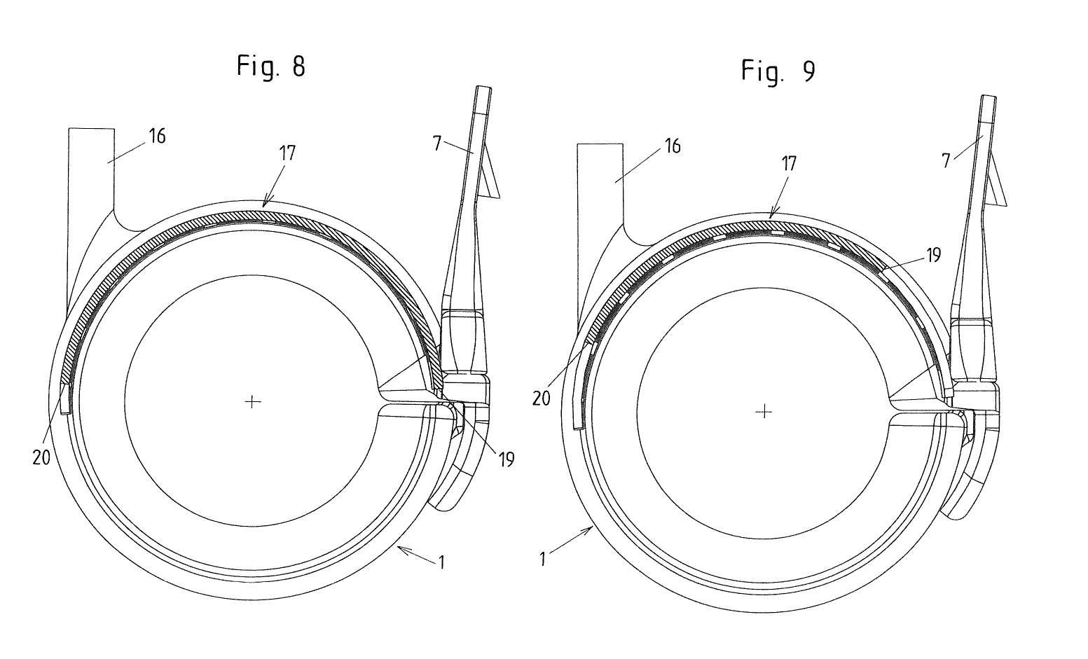

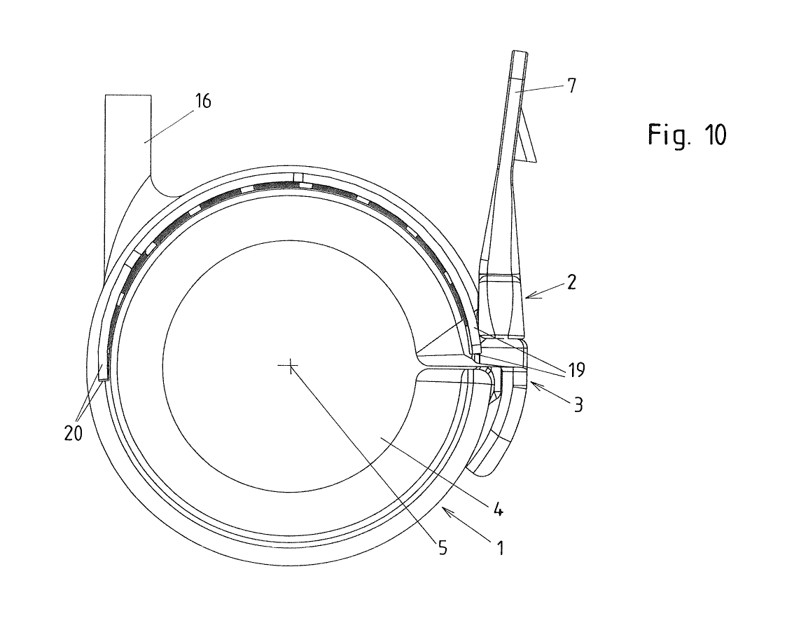

FIG. 7 shows a cross section lying in the area b1. FIG. 8 shows a cross section lying in the area b2. FIG. 9 shows a cross section, lying in the area b3, through the support part 17.

The reduction in the extent of the support part 17 about the central axis 5 as its distance from the mid-plane 6 increases is provided by at least one obliquely extending portion of one of the longitudinal edges 19, 20 of the support part 17, which edges delimit the extent of the support part 17 about the central axis 5, i.e. in the circumferential direction. A stepped profile would also be conceivable and possible.

The extent of the support part 17 in the direction of the central axis 5 is delimited, at the end of the support part 17 remote from the band part 1, by a side edge 21 of the support part 17, which side edge 21 here lies in a plane parallel to the mid-plane 6. It is also conceivable and possible for the side edge 21 to have a profile that is oblique or stepped at least in some parts.

Laterally with respect to the stomach, the support part 17 can be secured to the posterior wall of the abdominal cavity, preferably by sewing. For this purpose, in the areas of the transition between the side edge 21 of the support part 17 and the longitudinal edges 19, 20 of the support part 17, suture eyelets 22, 23 of the support part 17 are provided (the obliquely extending edge areas of the support part 17 are here assigned to the longitudinal edges 19, 20, since their inclination in relation to the central axis 5 is at least 45°).

Moreover, suture eyelets 24, 25 are expediently formed in the area of the transition between the obliquely extending portions of the longitudinal edges 19, 20 and those portions of the longitudinal edges 19, 20 extending parallel to the central axis 5.

Additional suture eyelets can be provided, particularly in the area of the longitudinal edges 19, 20.

To be able to take up the forces exerted on the support part 17 by the stomach, the support part 17 is preferably strengthened in the area of the suture eyelets 22-25, for example by inserts provided in these areas.

The circumferential extent of the support part 17 at the location where it issues from the band part 1, that is to say at the start of its extent away from the band part 1, is preferably in the range of 4.5 cm to 10 cm. The circumferential extent of the support part 17, at the end thereof remote from the band part 1, is preferably in the range of 2.5 cm to 7 cm.

In the illustrative embodiment shown, the radius of curvature of the support part 17 about the central axis 5 is constant along its extent in the direction of the central axis 5 and corresponds to the radius of curvature of the band part 1, with respect to the state of the band part 1 when closed to form a ring. The radius of curvature of the support part 17 in the area of its end remote from the band part 1 could also be smaller, for example at least 30% smaller, than the radius of curvature in the area of its end connected to the band part 1.

A convex design of the support part 17 would also be conceivable and possible, in which case the support part 17, in a middle area of its extent in relation to the direction of the central axis 5, and at least over part of its circumferential extent about the central axis 5, has a greater distance from the central axis 5 than in areas that have a smaller distance or a greater distance from the band part 1 than the middle area. Thus, the radius of curvature of the support part 17 in the middle area, at least over part of its circumferential extent about the central axis 5, is smaller than in the areas adjacent on both sides. It is possible in this way to achieve good adaptation to the shape of the stomach.

The area of the longitudinal extent of the band part 1 above which the support part 17 protrudes laterally therefrom adjoins that end of the band part 1 on which the second closure part 3 is arranged. The other end of the band part 1, where the first closure part 2 is arranged, is adjoined by a portion of the longitudinal extent of the band part 1 without support part 17 protruding laterally therefrom.

FIG. 13 is a schematic view of the device according to the invention in the state when applied to the stomach 26 of a patient. The band part 1 is applied around the stomach 26 and is closed by means of the closure mechanism 2, 3 to form a ring. In order to place the band part 1 around the stomach, a tunnel (channel) is first prepared which extends between the posterior wall of the stomach and the posterior wall of the abdominal cavity and through which has been engaged the first end of the still open band part 1 that is adjoined by the portion of the band part 1 designed without a support part 17 protruding laterally therefrom.

After the band part 1 has been placed around the stomach 26 and closed, the support part 17, in the area lying proximally from the band part 1, lies on the anterior ventral wall of the stomach. Then, laterally with respect to the stomach and on both sides thereof, the support part 17 is connected, in particular by sewing via the suture eyelets 22-25, to the posterior wall 27 of the abdominal cavity, of which a portion is shown schematically in FIG. 13.

The area of the oesophagogastric junction can also be supported by the support part 17.

On the side of the band part 1 away from the support part 17, it would be possible in principle for a further support part to protrude from the band part 1. This further support part would then lie on the outside of the stomach, in an area of the stomach lying distally from the band part 1. A further support part of this kind, in particular with a net-like design, could be formed by a separate part or could be made in one piece with the support part 17.

### KEY TO THE REFERENCE NUMBERS

1 band part
2 first closure part
3 second closure part
4 opening
5 central axis
6 mid-plane
7 continuation
8 locking projection
9 web
10 preliminary locking projection
11 pulling loop
12 hollow chamber
13 inner area
14 outer area
15 injection port
16 connector tube
17 support part
18 through-opening
19 longitudinal edge
20 longitudinal edge
21 side edge
22 suture eyelet
23 suture eyelet
24 suture eyelet
25 suture eyelet
26 stomach
27 posterior wall

1. Device for treating obesity, comprising a band part (1), which can be applied in a ring shape around a stomach, said band part (1) having a closure mechanism (2, 3) with which the band part (1) can be closed to form a circumferentially closed ring having an opening (4) with a central axis (5), and a support part (17), which is connected to the band part (1) and which protrudes from the band part on one side and provides support for a stomach area lying proximally with respect to the band part (1) in an applied position, wherein the support part (17) extends by less than 270° about the central axis (5) in a sectional plane for each cross section that passes through the support part (17) and lies parallel to a mid-plane (6) in which the band part (1) closed to form a ring lies and which extends centrally through the band part (1).

2. Device according to claim 1, wherein the band part (1) has a first portion of a longitudinal extent thereof that adjoins a first end of the band part (1) and that has a length amounting to at least one third of a total length of the band part (1), and has a second portion of its longitudinal extent that adjoins a second end of the band part (1) and that has a length amounting to a remaining length of the band part (1), wherein the support part (17) protrudes from the side of the band part (1) only over the second portion of the longitudinal extent of the band part (1).

3. Device according to claim 2, wherein the support part comprises a first area (b1) which lies at a distance from the mid-plane (6) amounting to less than one third of a maximum distance (a) of a side edge (21) of the support part (17) from the mid-plane (6), a second area (b2) which lies at a distance of the support part (17) from the mid-plane (6) that amounts to more than one third and less than two thirds of the maximum distance (a) of the side edge of the support part (17) from the mid-plane (6), and a third area (b3) which lies at a distance of the support part (17) from the mid-plane (6) that amounts to more than two thirds of a maximum distance (a) of the side edge (21) of the support part (17) from the mid-plane (6), wherein in the third area (b3) of the support part (17) a greatest extent of the support part (17) about the central axis (5) is smaller than a greatest extent of the support part (17) about the central axis (5) in the first area (b1) of the support part (17), which lies at a distance from the mid-plane (6) amounting to less than one third of the maximum distance (a) of the side edge (21) of the support part (17) from the mid-plane (6).

4. Device according to claim 3, wherein in the second area (b2) of the support part (17) a greatest extent of the support part (17) about the central axis (5) is smaller than the greatest extent of the support part (17) about the central axis (5) in the first area (b1) of the support part (17).

5. Device according to claim 4, wherein in the third area (b3) of the support part (17) the greatest extent of the support part (17) about the central axis (5) is smaller than the greatest extent of the support part (17) about the central axis (5) in the second area (b2) of the support part (17).

6. Device according to one of claims 1, wherein the support part (17) has a multiplicity of through-openings (18) distributed across an expanse thereof.

7. Device according to claim 6, wherein the support part (17) has a net-like design.

8. Device according to claim, wherein the support part (17) is adhesively bonded to the band part (1).

9. Device according to claim 1, wherein in an area of transitions between longitudinal edges (19, 20) of the support part (17), which delimit an extent of the support part (17) about the central axis (5), and a side edge (21) of the support part (17), which at an end of the support part (17) remote from the band part (1) delimits an extent of the support part in a direction of the central axis (5), the support part (17) is designed such that it can be connected to the posterior wall of an abdominal cavity.

10. Device according to claim 9, wherein the support part (17) has suture eyelets (22, 23, 24, 25) adapted for sewing it onto the posterior wall (27) of the abdominal cavity on both sides of a stomach (26).

11. Device according to claim 3, wherein the greatest extent of the support part (17) about the central axis (5) in the third area (b3) is at least 10% smaller than the greatest extent of the support part (17) about the central axis (5) in the first area (b1) of the support part (17).

12. Device according to claim 4, wherein the greatest extent of the support part (17) about the central axis (5) in the second area (b2) is at least 10% smaller than the greatest extent of the support part (17) about the central axis (5) in the first area (b1) of the support part (17),

13. Device according to claim 5, wherein the greatest extent of the support part (17) about the central axis (5) in the third area (b3) is at least 10% smaller, than the greatest extent of the support part (17) about the central axis (5) in the second area (b2) of the support part (17).

14. Device according to claim 8, wherein the support part (17) is adhesively bonded to the band part (1) in an area of one of the longitudinal edges of the band part (1).

15. Device according to claim 9, wherein the support part (17) is designed such that it can be sewn to the posterior wall of an abdominal cavity.

16. Device according to claim 2, wherein the length of the first portion of the longitudinal extent of the band part (1) amounts to at least two fifths of the total length of the band part (1).
